(19) United States
(12) Patent Application Publication
(10) Pub. No.: US 2002/0164800 A1
(43) Pub. Date: Nov. 7, 2002

(54) Retroviral vector

(76) Inventors: Clayton A. Smith, Tampa, FL (US); Eli Gilboa, Durham, NC (US)

Correspondence Address: NIXON & VANDERHYE P.C., 8th Floor, 1100 North Glebe Road, Arlington, VA, 22201, US

(21) Appl. No.: 10/059,265

(22) Filed: Jan. 31, 2002

Related U.S. Application Data
(60) Provisional application No. 60/265,123, filed on Jan. 31, 2001.

(51) Int. Cl. C12N 15/867; C12N 7/00; C12N 7/01; C12N 15/09; C12N 15/70; C12N 15/86

(52) U.S. Cl. 435/456; 435/320.1; 435/235.1

(57) ABSTRACT

The present invention relates, in general, to a retroviral vector and, in particular, to a Moloney murine leukemia virus-based retroviral vector. The invention further relates to methods of introducing genetic elements into cells, including mammalian cells, using such a vector.

This application claims priority from U.S. Provisional Application No. 60/265,123, filed Jan. 31, 2001, the entire content of which is incorporated herein by reference.

### TECHNICAL FIELD

The present invention relates, in general, to a retroviral vector and, in particular, to a Moloney murine leukemia virus-based retroviral vector. The invention further relates to methods of introducing genetic elements into cells, including mammalian cells, using such a vector.

### BACKGROUND

Retroviruses have been used for some time as vectors to mediate gene transfer into eucaryotic cells. The suitability of retroviruses for gene transfer results from their mode of replication. By replacing vital genes of the virus with a gene of interest and utilizing the efficient viral infection process, the gene is transferred into the target cells as if it were a viral gene (most of the widely used viral vectors are derived from the genome of the Moloney Murine Leukemia Virus (MoMuLV)). More specifically, using standard recombinant DNA techniques, portions of the viral DNA (i.e., internal sequences) can be combined with the gene of interest. The remaining retroviral DNA is called the vector and includes the two ends of the viral genome, which are terminally redundant and “designated long terminal repeats” (LTRs). This and immediately adjacent regions of the viral genome contain important cis functions necessary for the replication of the virus, for example, the viral packaging signal. The deleted sequences which can be replaced with the foreign gene or genes, encode proteins that are necessary for the formation of infectious virions. These proteins, although necessary for the replication of the virus, can be complemented in trans if the cell contains another virus expressing the gene products missing in the vector. The hybrid DNA can be introduced into specially engineered cells by standard DNA transfection procedures. These cells, called packaging cells, harbor a retrovirus defective in a cis function. The RNA of such cells cannot encapsidate into a virion but can express all the viral proteins and can, therefore, complement the functions missing in the incoming vector DNA. The vector DNA is then transcribed into a corresponding RNA which is encapsulated into a retrovirus virion and secreted. The actual gene transfer takes place at this point: the virus is used to infect target cells, and through the efficient viral infection process, the foreign gene is inserted into the cell chromosome as if it were a viral gene.

Retroviral based gene transfer is a promising technique for two principal reasons. First, it is highly efficient. At present, retroviral based gene transfer is the only system available for use in cases where it is necessary to introduce the gene of interest into a large proportion of target cells. This is in contrast to other gene transfer systems, such as DNA transfection, protoplast fusion and electroporation. Second, retroviral vectors have a broad host range, which enables genes to be introduced not only into monolayers of cultured cells but also into suspension-grown lymphoid and myeloid cells and hemopoietic stem cells present in bone marrow population.

A limitation of retroviral vectors is the requirement for multiple manipulations. When using DNA transfection, electroporation or protoplast fusion, the DNA fragment carrying the gene of interest is directly introduced into target cells, whereas in using retroviral vectors the gene of interest is first inserted into a retroviral vector and converted into a virion before the actual gene transfer takes place. It is now quite simple to insert a gene into a retroviral vector, obtain recombinant virus, infect target cells and express the foreign gene. Maximizing the efficiency of the process, however, is more difficult. It is this problem that is addressed by the present invention.

### SUMMARY OF THE INVENTION

The present invention relates to a Moloney Murine Leukemia Virus-based retroviral vector and to a method of introducing genetic elements into cells using same. In a preferred embodiment, the vector utilizes an extended gag sequence in order to optimize packaging of the viral genome and improve vector titer. Wild type splice signals can be present and the viral env ATG can be used as the start codon for transgene cDNA in order to optimize viral RNA processing and transgene expression. Disruption of the Pr65 gag ORF with a stop codon minimizes the possibility of encoding gag peptides that can contribute to vector immunogenicity and toxicity. MoMLV env sequences that can contribute to vector immunogenicity and toxicity are essentially eliminated. A multiple cloning site can be included within the 3′ LTR U3 region for development of, for example, double copy vectors. Modular construction facilitates modifications to both internal and 5′ or 3′ regions.

Objects and advantages of the present invention will be clear from the description that follows.

### BRIEF DESCRIPTION OF THE DRAWINGS

FIG. 1. The architecture of the LUV vector. LTR=long terminal repeat, SD=splice donor, Y=the packaging sequence, ATG∂ATG=mutation in the gag ORF to eliminate translation of gag peptides, SA=splice acceptor, ATG=env ATG cloned in frame with the marker of therapeutic transgene cDNA. MCS is the multiple cloning site in the 3′ LTR.

FIG. 2. Fold expansion of total cells and erythroid lineage cells derived from UCB grown under serum free-conditions.

FIGS. 3A and 3B. FIG. 3A. Dot plots of transduced cells at days 8, 15, and 22, with table comparing red cell lineage purity of transduced and non-transduced populations. FIG. 3B. Transduced and non-transduced populations stained with Wright-Giemsa alone (top) or immunoflourescent staining superimposed on Wright-Giemsa staining (bottom). NGFR is stained with PE (red) and the erythroid cell are stained with FITC (green).

FIG. 4. Percent of erythroid cells transduced with NGFR.

FIGS. 5A-5D. Erythroid lineage cells derived from umbilical cord blood mononuclear cells. FIG. 5A. Representative morphology of erythroid cells generated from UCB. Wright Giemsa staining was then performed on days 1, 3, 8, 15, and 22. FIG. 5B. Representative FACS analysis of erythroid cells generated from UCB. Cells were stained at progressive time points with the erythroid cell specific antibody E6. The percentage of E6 expressing cells is presented above the M1 cursor. (The data in panels A and B is representative of 5 experiments.) FIG. 5C. Fold expansion of total cells and erythroid lineage cells generated from UCB. The data is the mean of 5 experiments. FIG. 5D. Cellulose acetate electrophoresis analysis of erythroid cells generated from UCB. Y axis indicates migration pattern for various hemoglobin types. The data is representative of 3 experiments.

FIGS. 6A and 6B. Peripheral Hb SC mononuclear cell cultures. FIG. 6A. FACS analysis of erythroid cells generated from Hb SC mononuclear cells stained at progressive time points with the erythroid cell specific antibody E6. The percentage of E6 expressing cells is presented above the M1 cursor. Data is representative of 3 experiments. FIG. 6B. Fold expansion of total cells and erythroid lineage cells generated from Hb SC mononuclear cells. The data is the mean of 3 experiments.

FIG. 7. The Luv vector backbone compared to the Moloney Murine Leukemia Virus (MoMLV) genome. LTR=long terminal repeat, SD=splice donor, Y=the packaging sequence, ATG [symbol] TAG=mutation in the gag open reading frame to eliminate translation of gag peptides, SA=splice acceptor, ATG=envelope start codon cloned in frame with the marker or therapeutic transgene (cDNA), TAG=envelope stop codon, ppt is the polypurine tract, MCS is the multiple cloning site in the 3′ LTR.

FIGS. 8A-8D. Transduction of NIH3T3 cells with LuvGM and LuvNM. FIGS. 8A and 8C demonstrate the background staining for gfp and anti-NGFR respectively. FIGS. 8B and 8D demonstrate the transduction efficiency with LuvGM and LuvNM respectively. The percentage of gene marked cells is presented above the M1 cursor.

FIG. 9. Immunofluorescence microscopy of erythroid cells generated from UCB and transduced with LuvNM. T=transduced, NT=nontransduced controls. At each indicated time point, the upper panels are stained with Wright Giemsa (W-G) and directly below them are the identical fields stained with both W-G and anti-NGFR-PE (red), anti-E6 FITG (green). Similar results were obtained with LuvGM. Data is representative of 5 experiments.

FIGS. 10A and 10B. FACS analysis of erythroid cells generated from UCB transduced with LuvNM. Erythroid cells transduced with LuvNM and then analyzed at progressive time points for staining with the erythroid specific antibody E6 and anti-NGFR. FIG. 10A is the mock infected controls, FIG. 10B is the LuvNM transduced cells. Data is representative of 5 experiments.

FIGS. 11A-11C. Transduction of UCB derived cells. FIG. 11A. The mean percentage of UCB derived erythroid lineage cells expressing NGFR. FIG. 11B. Comparison of total cell expansion from transduced and non-transduced samples based on viable cell counts. FIG. 11C. Comparison of erythroid cell expansion from transduced and non-transduced samples based on viable cell counts multiplied by the proportion of cells staining positive for E6. All data is the mean of 5 experiments.

FIGS. 12A-12C. Transduction of Hb SC PBMC derived cells. FIG. 12A. The mean percentage of Hb SC PBMC derived erythroid lineage cells expressing NGFR. FIG. 12B. Comparison of total cell expansion from transduced and non-transduced samples. FIG. 12C. Comparison of erythroid cell expansion from transduced and non-transduced samples. All data is the mean of 3 experiments.

### DETAILED DESCRIPTION OF THE INVENTION

The present invention relates to a retroviral vector that can be used to introduce into a eucaryotic cell a desired nucleic acid sequence. The instant vector (designated “LUV”) represents a derivative of the MoMuLV and is characterized by a similar transduction efficiency. The LUV vector system consolidates many of the useful elements of retroviral vectors, including high titer, efficient expression of foreign genes and safety. Further, the construction of the vector is such that each component thereof can be removed and replaced in a modular fashion.

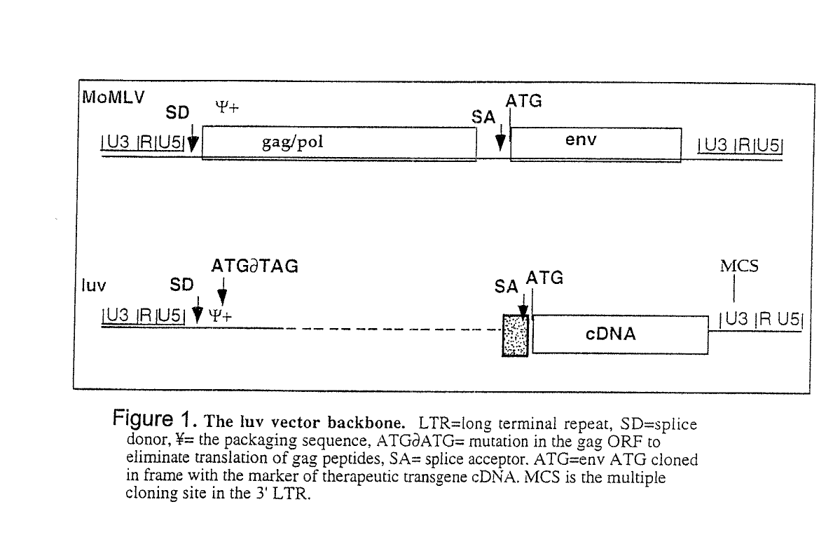

The design of a preferred embodiment of the LUV vector, and its relationship to the parental MoMuLV sequence, is shown in FIG. 1. As shown in FIG. 1, the preferred vector includes an extended N2-derived packaging signal for high titer (for a description of N2 see Armentano et al, J. Virol. 61:1647 (1987)). The preferred vector also includes wild type splice signals and the viral env ATG can be utilized as the start codon for transgene cDNA in order to optimize viral RNA processing and transgene expression. Multiple cloning sites can be present in the 3′ LTR, for example, Self Inactivating (SIN) and Double Copy (DC) vector design (see Yu et al, Proc. Natl. Acad. Sci. USA 83:3194 (1986) and U.S. Pat. No. 5,658,775, respectively). Further, disruption of Pr65 gag ORF minimizes expression of vector-derived peptides and thus reduces vector immunogenicity and/or toxicity. Removal of 3′ untranslated sequences reduces recombination and therefore generation of replication competent virus (RCV). The LUV vector can include a second polyadenylation signal (3′ to the LTR) to prevent read through and to increase titer. Replacement of the viral promoter with a heterologous promoter can increase vector RNA expression and viral titer and reduce recombination and RCV formation.

The vector of the invention can be used to introduce into cells virtually any sequence. The sequence can encode an RNA sequence, such as antisense RNA, or encode a polypeptide or protein of interest, preferably, a mammalian polypeptide or protein (e.g., adenosine deaminase (ADA)). The antisense RNA can be an RNA sequence that is complementary to a nucleotide sequence encoded by a pathogen, such as a bacteria, parasite or virus, e.g., the Human Immunodeficiency Virus (HIV). Alternatively, the sequence can encode an RNA that is the recognition sequence for a DNA or RNA binding protein. The sequence can also encode a selectable or identifiable phenotypic trait, such as resistance to antibiotics, e.g., ampicillin, tetracycline, and neomycin, and/or can comprise a non-selectable gene (e.g., green fluoresence protein).

Methods suitable for use in preparing the retroviral vector of the present invention are described in Example 1.

Additionally, this invention relates to a method of producing an infectious viral particle useful for introducing into a eucaryotic cell DNA encoding the desired nucleic acid. The method comprises introducing the retroviral vector described above into a suitable packaging cell line (e.g., AM12, PG13), culturing the packaging cell line under conditions such that the viral particle is formed within, and excreted by, the packaging cell line, and recovering the viral particle from the cell culture supernatant. This invention also encompasses a virion produced by such a method.

This invention also relates to a method of introducing into a eucaryotic cell the retroviral vector of the invention. The method can comprise infecting the target cell with the viral particle produced by the method described above under conditions such that the vector is incorporated into the chromosomal DNA of the eucaryotic cell. Either ex vivo or in vivo gene transfer can be used. In one embodiment, the eucaryotic cell is a mammalian cell, either an epithelial cell or fibroblast, e.g., a hepatocyte or lymphocyte, or a hemopoietic stem cell, advantageously, a human cell. Methods of infection and methods of detecting the presence of the encoded products are also well known in the art (Phillips et al, Nature Med. 2:1154 (1996)).

The invention further relates to a pharmaceutical composition comprising a therapeutically or prophylactically effective amount of a retroviral vector or infectious particle as well as a mammalian cell of the invention as a therapeutic agent. Such a pharmaceutical composition can be produced in a conventional manner. In particular, a retroviral vector or an infectious particle as well as a mammalian cell of invention can be combined with appropriate substances well known in the art, such as a carrier, diluent, adjuvant or excipient. The particular formulation of the pharmaceutical composition depends on various parameters, for example, the protein of interest to be expressed, the desired site of action, the method of administration and the subject to be treated. Such a formulation can be determined by those skilled in the art and by conventional knowledge.

Vectors of the present invention can be used in gene therapy regimens to effect the transfer of genes encoding molecules of therapeutic importance (Kozarsky et al, Current Opin. Genet. Develop. 3:499-503 (1993); Rosenfeld et al, Cell 68:143-155 (1992); Rogot et al, Nature 361:647-650 (1993); Ishibashi et al, J. Clin. Invest. 92:883-893 (1993); Tripathy et al, Proc. Natl. Acad. Sci. USA 91:11557-11561 (1994). Such genes include adenosine deaminase, glucocerebrosidase, β-globin and CD18.

Protocols suitable for use in administering the vectors of the invention include direct administration (e.g., by injection) to target tissue, intravascular administration, and catheter-based administration, for example, when the target is vascular tissue. Tissue specific expression of the gene transferred can be facilitated using tissue specific promoters (e.g., Ig heavy chain promoter for B-cells).

The invention also relates to a method of treating a genetic disorder or a disease induced by any pathogenic gene, such as cancer or a virally-induced disease, which comprises administering a therapeutically effective amount of a retroviral vector or an infectious particle as well as a mammalian cell of the invention to a subject in need of treatment. (See for details Karlsson, Blood 78:2481 (1991)).

Certain aspects of the present invention are described in greater detail in the non-limiting Examples that follow.

### EXAMPLE 1

### Retroviral Vector Construction and Analysis

The LUV vector backbone was constructed by using PCR to clone modular elements from a molecular clone of proviral Moloney murine leukemia virus. Mutagenesis of the PCR fragments via mismatched primer and add-on of specific sequences to the ends of PCR fragments was used for directional assembly of proviral plasmids. Overlapping PCR was utilized for site specific mutagenesis and for precise promoter placement with respect to transcriptional start points.

Hot start PCR reactions were performed in 100 μl of a mixture containing 100 μM dNTPs, 0.5 μM each primer (see primers given in Example 3), 10 ng template DNA, 10 μl 10XPfu buffer and 2.5 units Pfu DNA polymerase (Stratagene). The reaction was initially denatured by incubating at 95° C. for four minutes followed by two cycles of 95° C.-30 sec, 50° C.-30 sec, 72° C.-2 min, 25 cycles of 95° C.-30 sec, 60° C.-30 sec, 72° C.-2 min, then held at 72° C. for seven minutes. PCR products were purified using Centricon-100 columns (Amicon) according to the manufactures directions. Each fragment was individually ligated into the plasmid puc19 and sequenced. A multiple cloning site (5′ctagcgtacggcatgcatgcacgcgtctctcgagctccgcgg, 5′ctagccgcggagctcgagagacgcgtgcatgcatgccgtacg) was introduced into the unique NheI site within the U3 region of fragment 3 for subsequent generation of double copy vectors. Individual fragments were then combined and ligated into the unique Hind III/Eco RI site of pBR322 in which BamHI site was destroyed to generate the luvM (LUV plus multiple cloning site for generation of ‘double copy’ vectors) proviral plasmid.

LuvNM retroviral vector was generated by inserting NGFR (nerve growth factor receptor) cell surface selectable marker into one of the cloning sites of luvM. A recombinant luvNM retrovirus producer cell line was produced by cotransfection of luvNM and Tkneo plasmids into the ecotropic packaging cell line E86 with G418 selection (0.8 mg/ml Gibco-BRL) for 10 days (Bank). Cell free viral supernatant was harvested and used to infect the amphotropic AM12 packaging line (Bank). (AM12 luvNM producers were sorted to purity by flow cytometry.) End point dilution titers of bulk AM12 derived luvNM retroviral supernatant approached 5×10⁶ TU/ml and tested negative for replication competent retrovirus by a marker rescue assay.

A variety of LUV based vectors have been analyzed for titer and transgene expression. In these vectors, expression of the marker genes NGFR and green fluorescent protein (GFP) have been utilized in the LUV vector series for rapid analysis of vector transduction efficiency and level of transgene expression. LUV vectors constructed and analyzed include:

| | |
|---|---|
| luv M | (luv plus multiple cloning site for |
| | generation of ‘double copy’ vectors) |
| luvN | (luv plus NGFR cell surface selectable |
| | marker) |
| luvNM | (luvN plus multiple cloning site for |
| | generation of ‘double copy’ vectors) |
| luvNMpA | (luvNM plus addition of synthetic poly A |
| | signal) |
| luvGM | (luvM plus GFP marker gene) |
| cluv | (luv with MoMLV U3 viral promoter |
| | replaced by huCMV-IE promoter) |
| cluvM | (cluv plus multiple clonining site for |
| | generation of ‘double copy’ vectors) |
| cluvN | (cluv plus NGFR cell surface selectable |
| | marker) |
| cluvNM | cluvN plus multiple cloning site for |
| | generation of ‘double copy’ vectors) |
| cluvNMpA | (cluvMN plus addition of synthetic poly |
| | A signal) |
| cluvGM | (cluvM plus GFP marker gene) |

Expression of both NGFR and GFP from these vectors was very high in NIH3T3 cells, primary human T-lymphocytes and primary human CD34+ hematopoietic progenitors Specifically, titers of these vectors ranged from 5×10⁵ to 4×10⁶ infectious units/ml on NIH3T3 cells with infection efficiencies of 10-30% in primary T-lymphocytes and hematopoietic progenitors. In comparative studies, luvNM and luvGM appeared to yield titers and transgene expression equivalent to, or better than, other available MoMLV based vectors including kat (Finer, Blood 83:43 (1994)), MFG (Proc. Natl. Acad. Sci. 92:6728), N2, and LXSN (Miller, Biotechniques 7:980) in NIH3T3 cells.

### EXAMPLE 2

### Transduction of Erythrocyte Precursors

Experimental

Isolation and culture of mononuclear cells from umbilical cord blood and peripheral blood of patients with sickle cell anemia. Peripheral blood intended for disposal was obtained in (ACD) from patients with hemoglobin sickle cell (SC) disease undergoing scheduled phlebotomy in accordance with IRB protocol on discarded materials. Umbilical cord blood intended for disposal was collected from labor and delivery into citrate-phosphate-dextrose anticoagulant (Abbott Laboratories, North Chicago, Ill.). Mononuclear cells were isolated by Ficol-Hypaque gradient separation (American Red Cross, Washington D.C.), and suspended at a concentration of 1×10⁶ cells per milliliter in serum free conditions consisting of Iscove's Modified Delbecco's Medium with 1% bovine serum albumin, 10 μg/ml insulin, 200 μg/ml transferrin (BIT9500 media, Stem Cell Technologies, Vancouver, BC), 40 μg/ml low density lipoprotein (Sigma), 2 μM glutamine (Life Technologies, Grand Island, N.Y.), and 5×10⁻⁵M β-mercaptoethanol (Life Technologies), supplemented with Flt-3 ligand (25 ng/ml, Immunex, Seattle Wash.), IL-3(2.5 ng/ml, R&D Systems, Minneapolis, Minn.), Erythropoeitin (1 μu/ml, R&D Systems, Minneapolis, Minn.). Cells were incubated at 37° C. 5% CO₂ overnight, then transferred to fresh plates to eliminate adherent cells.

Retroviral transduction of erythrocyte precursors. On day 3, cells were counted and the volume was adjusted to deliver 5×10⁵ cells in 375 μl of serum free media. Cells were transferred to a 24 well plate coated with 25 μg/ml RetroNectin (PanVera Corp., Madison Wis.). LuvNM retroviral vector supernatant (prepared as described in Example 1) was added in a 3:1 cell to supernatant ratio by volume and incubated at 37° C. 5% CO₂. An equal volume of retroviral supernatant was added on days 4 and 5.

Staining and analysis of transduced erythrocyte precursors. Samples were counted weekly and media was added to maintain the cell concentration at 5×10⁵ cells per milliliter. An aliquot of cells was analyzed by FACS analysis and immunofluorescent microscopy on days 1, 3, 8, 15, and 22. Briefly, the cells were washed with PBS, and resuspended in 100 μl versene (Gibco BRL) with 4% fetal calf serum. Cells were incubated with an antibody to red cell surface protein band 3, washed with PBS, stained with a secondary antibody conjugated to FITC (Jackson ImmunoResearch Lab. Inc., West Grove Pa.), washed, and stained with an NGFR monoclonal antibody conjugated with phycoerythrin. 7-AAD was added to exclude dead cells and all samples were run on a FACStar (BectonDickenson). An aliquot of the stained cells was cytocenterfuged (Shandon Lipshaw, Pittsburgh Pa.) and viewed under a fluorescent microscope. These slides were then stained with Wright-Giemsa using an automated stainer, and the images were superimposed.

Results

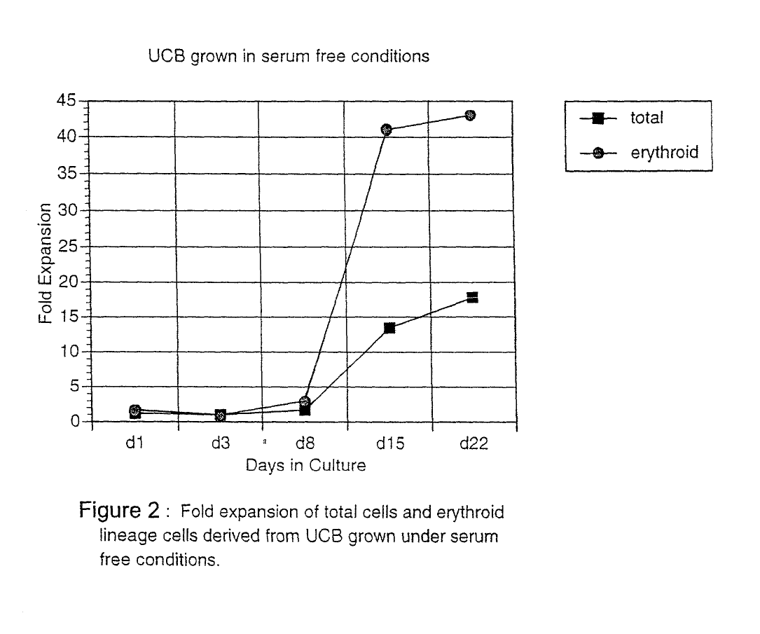

Erythrocyte precursors can be successfully generated in serum-free liquid culture. During the 22 days in culture, the percent of erythroid lineage cells increased from a mean of 39.8% on day number one, to almost 90% on day number 15, with a slight decline thereafter. Total cell expansion and erythroid lineage expansion are shown in FIG. 2. Umbilical cord blood showed an 18 fold expansion reaching a maximal number on day 22 and contained 88% erythrocytes or erythrocyte precursors on day 15.

Peripheral blood from patients with Hemoglobin SC disease showed a 12 fold expansion, reaching a maximal number on day 22, and contained 81% erythrocytes or erythrocyte precursors on day 15.

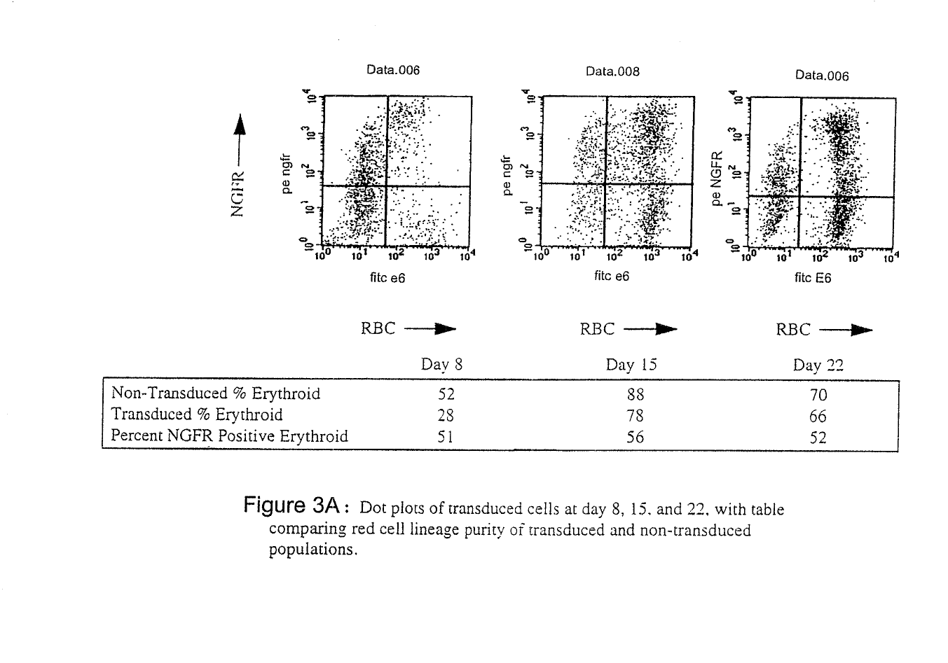

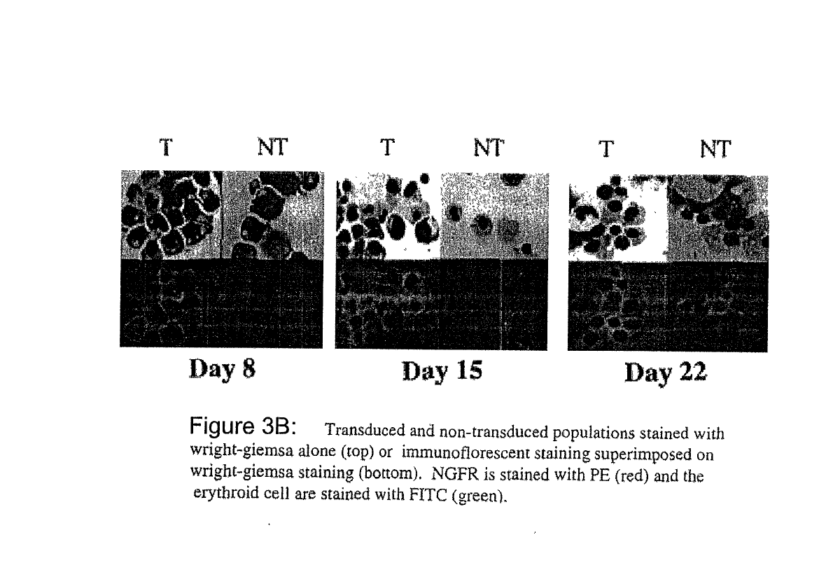

The LuvNM vector can efficiently transduce erythrocyte precursors and is at least as effective then the parent retroviral vector AM12MN. The luvNM vector did not alter the growth or purity of erythroid cells generated in serum free conditions, however it was capable of transducing greater then 50% of the erythroid cells (FIG. 3).

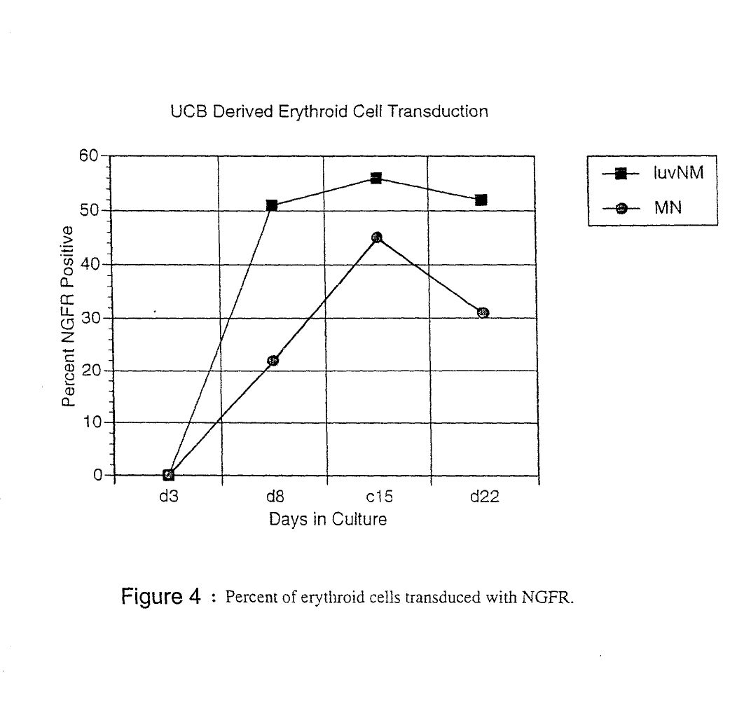

Both the AM12MN parent retrovirus, and the luvNM vector, were capable of transducing over 50% of the erythrocyte precursors FIG. 4. While the MN retrovirus reached a peak erythrocyte transduction efficiency on day 15 and then declined, the transduction efficiency with the luvNM retrovirus remained relatively constant over the 22 days.

### EXAMPLE 3

### Evaluation of Red Cell Transduction

This study was designed to develop procedures for evaluating gene transfer vectors in primary erythroid precursors generated from healthy donors and from persons with hemoglobinopathies. A single-step serum free culture system was developed for generating RBC precursors from mononuclear cells obtained from the umbilical cord blood of healthy neonates and from the peripheral blood of adults with Hb SC disease. In addition, retroviral vectors and techniques were developed which resulted in efficient gene transfer into the erythroid precursors generated in these culture conditions. A retroviral vector developed in the course of these studies was designed in a modular fashion so that future modifications designed to optimize the expression and stability of therapeutic transgenes could be readily performed. These methods and reagents for generating and efficiently transducing erythrocyte precursors from a widely available source provide a simple, quick, and effective means for evaluating vector constructs in clinically relevant cells.

Experimental Details

Retroviral Vector Construction:

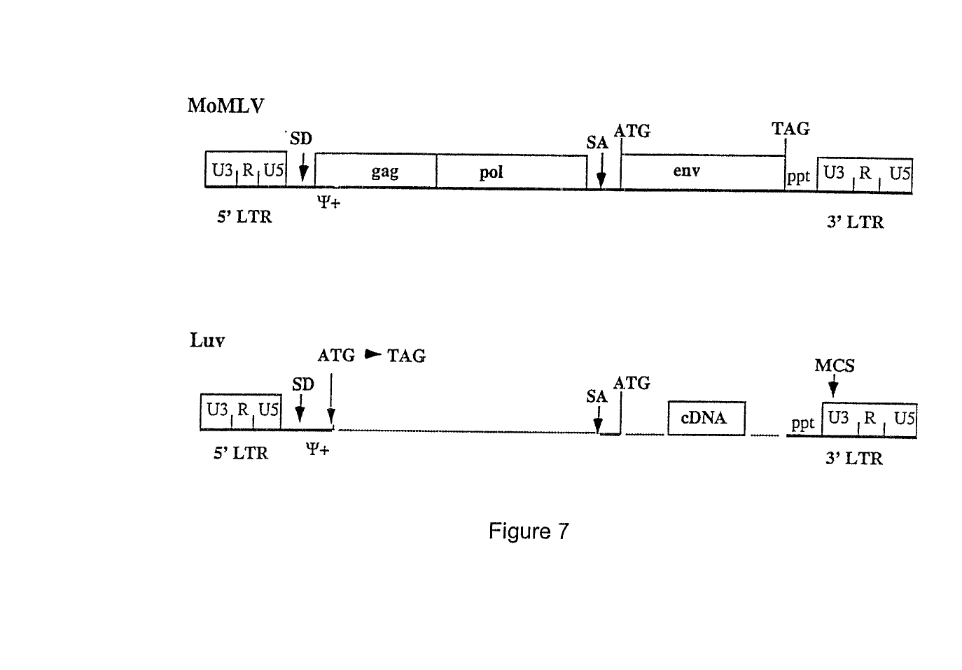

The LUV series of retroviral vectors was constructed by using PCR to clone modular elements from a molecular clone of proviral Moloney murine leukemia virus (MoMLV). The overall design of the Luv series and its relationship to the parental MoMLV sequence is depicted in FIG. 7. Directional assembly of proviral plasmids was accomplished using both mutagenesis of the PCR fragments via mismatched primers and by adding specific sequences to the ends of PCR fragments. The Luv vector is composed from 3 major fragments. Fragment 1 contains the entire 5′ LTR and extends to position 1041 of MoMLV with modification of the Pr65 gag initiating methionine to a TAG stop codon. Fragment 1 was generated in two steps from Fragments 1A and 1B using the PCR primer pairs described below. Fragment 2 extends from the wild type splice acceptor at position 5403 to the envelope start codon at position 5779 with modification of the A nucleotide at position 5775 to a C nucleotide. Fragment 3 begins with the envelope stop codon at position 7772 and extends through the entire 3′ LTR. The MoMLV sequence is printed in bold type and the restriction sites used for directional cloning of the three PCR fragments are underlined in

FIG. 7. The PCR primers used to generate the three fragments are listed below:

| | | |
|---|---|---|
| Fragment 1A: | (upstream) | 5′ ggcgcggcaagcttgaatgaaagaccccacctg 3′ |
| | (downstream) | 5′ ggtaacagtcttggccctaattctcagacaaatacag 3′ |
| Fragment 1B: | (upstream) | 5′ ctgtatttgtctgagaattagggccagactgttaccact 3′ |
| | (downstream) | 5′ ggcgcggcgagatctcatatggcgcctagagaagg 3′ |
| Fragment 2: | (upstream) | 5′ ggcgcggcagatcttatatggggca 3′ |
| | (downstream) | 5′ ggcgcggcccatggcagtctagaggatggtcc 3′ |
| Fragment 3: | (upstream) | 5′ ggcgcggcccatggcgcggatcccatagataaaaataaaag 3′ |
| | (downstream) | 5′ ggcgcggcgaattcaatgaaagacccccgctgacg 3′ |

Not start PCR reactions were performed in 100 ml of a mixture containing 100 mM dNTPs, 0.5 mM each primer, 10 ng template DNA, 10 ml 10X Pfu buffer and 2.5 units DNA polymerase (Stratagene, La Jolla, Calif.). The reaction was initially denatured by incubating at 95° C. for four minutes followed by two cycles of (95° C.-30 sec., 50° C.-30 sec, 72° C.-2 min.), 25 cycles of (95° C.-30 sec, 60° C.-30 sec, 72° C.-2 min.) then held at 72° C. for seven minutes. PCR products were purified using Centricon-100 columns (Amicon, Bedford, Mass.) according to the manufactures directions. Each fragment was individually ligated into the plasmid puc19 and sequenced. A multiple cloning site was introduced into the unique NheI site within the U3 region for subsequent generation of double copy vectors. Individual PCR fragments were combined and ligated into the unique Hind III/Eco RI site of pBR322 in which the BamHI site was destroyed to generate the LuvM proviral plasmid. To generate the LuvNM retroviral vector, the NGFR marker gene (McCowage et al, Experimental Hematology 26:288-298 (1998), Phillips et al, Nature Medicine 2:1154-1157 (1996)) was inserted into the NotI site in frame with the original Moloney envelope start codon (ATG). To generate the LuvGM vector, the GFP marker gene (Clontech, Palo Alto, Calif.) was also inserted into the NotI site. Recombinant amphotropic LuvNM and LuvGM virus were produced in the AM12 cell line and titered on NIH3T3 cells as previously described (McCowage et al, Experimental Hematology 26:288-298 (1998), Phillips et al, Nature Medicine 2:1154-1157 (1996)).

Isolation and culture of mononuclear cells from umbilical cord blood and peripheral blood of patients with Hb SC disease

Peripheral blood intended for disposal was obtained from patients with hemoglobin SC disease undergoing scheduled phlebotomy in accordance with IRB approved protocols. Umbilical cord blood was collected in accordance with IRB approved protocols from labor and delivery into citrate-phosphate-dextrose anticoagulant (Abbot Laboratories, North Chicago, Ill.). Mononuclear cells were isolated by Ficoll-Hypague gradient separation (American Red Cross, Washington, D.C.), washed three times with Delbecco's phosphate buffered saline (PBS) (Gibco BRL, Rockville, Md.) and suspended at a concentration of 1×10⁶ cells per milliliter in serum free conditions consisting of Iscove's Modified Dulbecco's Medium with 1% bovine serum albumin, 10 μg/ml insulin, 200 μg/ml transferrin (BIT95f00 media, Stem Cell Technologies, Vancouver, BC), 40 μg/ml low density lipoprotein (Sigma, St. Louis, Mo.), 2 μM glutamine (Life Technologies), and 5×10⁻⁵M β-mercaptoethanol (Life Technologies), supplemented with Flt-3 ligand (25 ng/ml, Immunex, Seattle, Wash.), IL-3 (2.5 ng/ml, R&D Systems, Minneapolis, Minn.), Erythropoietin (1 u/ml, R&D Systems, Minneapolis, Minn.), and Penicillin G (100 u/ml)/Streptomycin(100 μg/ml) (Gibco BRL). Cells were incubated at 37° in a 5% CO₂ incubator overnight, then transferred to fresh plates to eliminate adherent cells. Cells were then cultured in the same serum free media at 37° C. for further analysis and manipulation as described below.

Retroviral transduction of erythrocyte precursors

On day 3 of culture, cells were counted and the volume was adjusted so that the cell concentration was 5×10⁵ cells in 375 μl of serum free media. Cells were transfected to a 24 well plate previously coated with 25 μg/ml RetroNectin (PanVera Corp., Madison, Wis.). LuvNM or LuvGM retroviral vector supernatant was added in a 3:1 cell to supernatant ratio by volume and incubated at 37° in a 5% CO2 incubator. An equal volume of retroviral supernatant was also added on days 4 and 5.

Flow cytometric and morphologic analysis of cultured erythrocyte precursors

Samples were counted weekly and media was added to maintain the cell concentration at 5×10⁵ cells per milliliter. An aliquot of cells was analyzed by FACS analysis and immunofluorescent microscopy on days 1, 3, 8, 15, and 22. Briefly, the cells were washed with PBS, and resuspended in 100 ml versene (Gibco BRL) with 4% fetal calf serum. Cells were incubated with E6, an antibody to red cell surface protein band 3 (gift of Marilyn Telen, Durham, N.C.), washed with PBS, and then stained with a secondary antibody conjugated to FITC (Jackson ImmunoResearch Lab. Inc., West Grove Pa.). For gene transfer experiments, cells were then washed, and stained with an anti-NGFR monoclonal antibody conjugated with phycoerythrin. 7-AAD was added to exclude dead cells and all samples were run on a FACSCalibur (Becton-Dickenson, San Jose, Calif.). For morphologic analysis, an aliquot of the stained cells was cytocentrifuged (Shandon Lipshaw, Pittsburgh, Pa.) and fluorescent images were acquired using a digital video microscope (Olympus BX60 microscope, Optronics Engineering DEI-750 video camera, Scion Image 1.60 video capture software, NIH, Bethesda, Md.). The same slides were then stained with Wright-Giemsa using an automated stainer, the original cell field was located using bright field microscopy and the image was digitally acquired. The Wright-Giemsa and fluorescent images were then superimposed using Adobe Photoshop (Adobe Systems Incorporated, Seattle, Wash.). Cellulose acetate electrophoresis analysis of cultured cells was performed according to kit instructions modified for smaller sample sizes (Helena Laboratories, Beaumont, Tex.).

Results

Generation of erythrocyte precursors in serum-free liquid cultures from unfractionated UCB mononuclear cells and from peripheral blood mononuclear cells obtained from patients with hemoglobin SC disease.

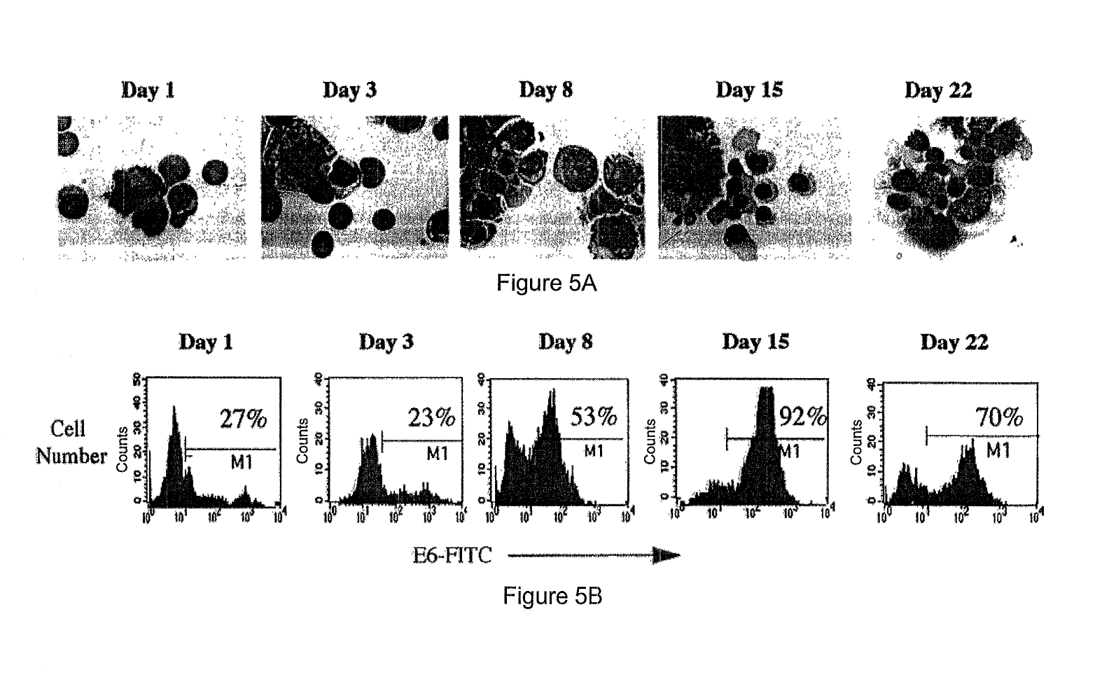

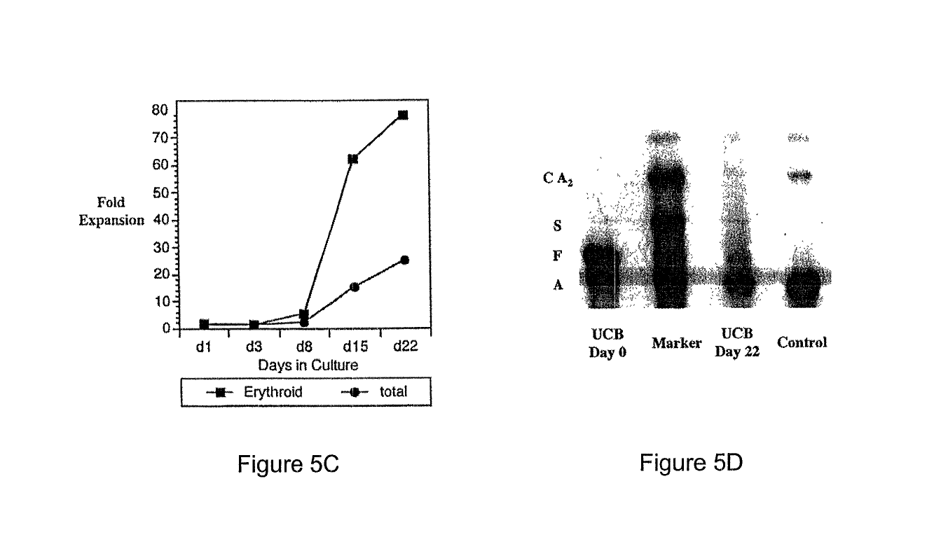

To facilitate evaluation of therapeutic genes in primary human RBCs, culture conditions were established for generating erythroid lineage cells directly from mononuclear cells. Fresh umbilical cord blood mononuclear cells were isolated and grown in liquid culture for three weeks in the presence of Flt-3-ligand, IL-3, and erythropoietin (Epo). This cytokine combination and culture duration was determined by comparing a variety of alternative culture conditions and incubation times. Morphologic analysis revealed that the majority of erythroid lineage cells present on day one of incubation were mature red blood cells (RBCs) (FIG. 5A). After eight days in culture, however, mature RBCs accounted for less than 20% of the total erythroid lineage, being replaced by more immature nucleated erythrocyte precursors (FIG. 5A). FACS analysis of cultured cells stained with the erythrocyte specific antibody E6 (Telen et al, Vox Sang. 52:236-243 (1987)) revealed that the percentage of erythroid lineage cells increased from a mean of 40% on day number one to a mean of 90% on day 15 in culture, with a slight decline thereafter (FIG. 5B). Overall, total cells expanded a mean of 25 fold (range 1.9-55 fold) and erythroid lineage cells expanded a mean of 78 fold (range 3-119 fold) (FIG. 5C). During the culture period, the hemoglobin content of the red cells shifted from primarily hemoglobin F at the start of incubation to primarily hemoglobin A at the end of the culture period (FIG. 5D).

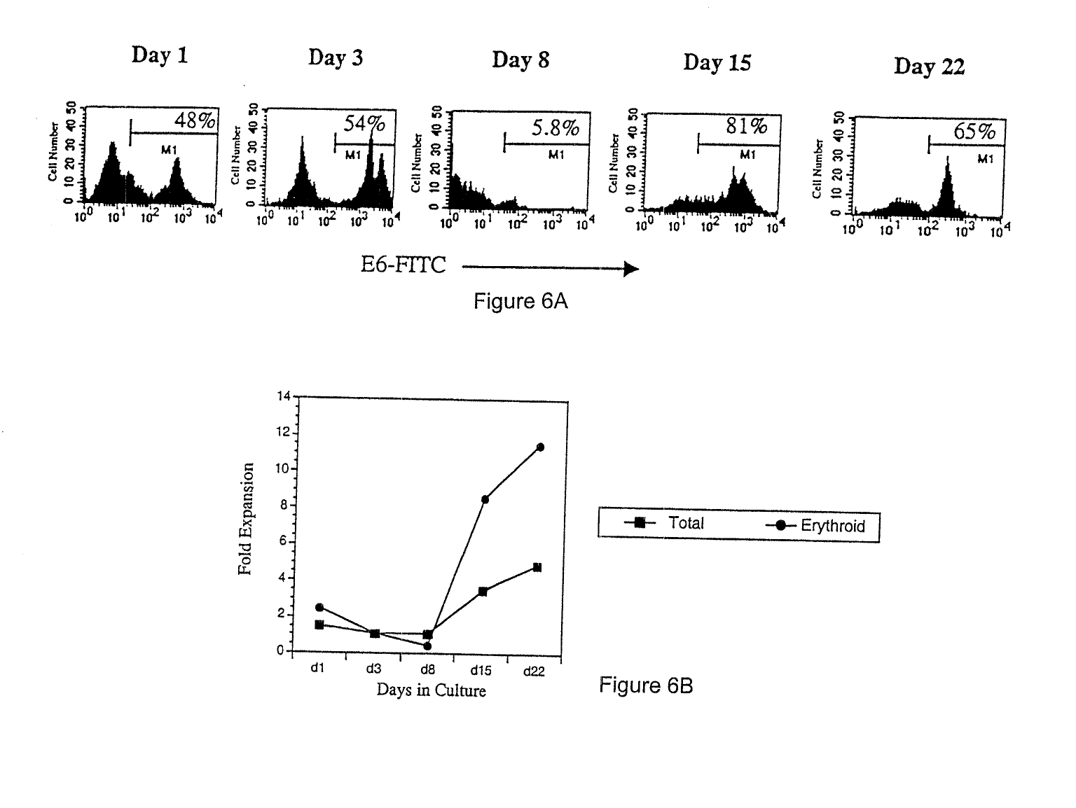

These same culture conditions were employed to generate RBC precursors from peripheral blood mononuclear cells (PBMCs) obtained from patients with hemoglobin SC disease undergoing therapeutic phlebotomy. Cells from 2 of 5 samples (both from the same individual) failed to expand during the culture period. In the other 3 samples, however, total cells expanded 4.8 fold (range 2.3 to 9.5) and erythroid cells expanded 11 fold (range 4.6 to 24) (FIG. 6B). The purity of erythroid lineage cells was similar to that seen with umbilical cord blood (FIG. 6A). Although considerable inter-individual variation exists, these data indicate that it is feasible to expand erythroid lineage cells using simple culture conditions directly from most UCB mononuclear cells and from many PBMCs obtained from patients with Hb SC disease.

Retroviral vector mediated gene transfer into RBC precursors generated from mononuclear cells

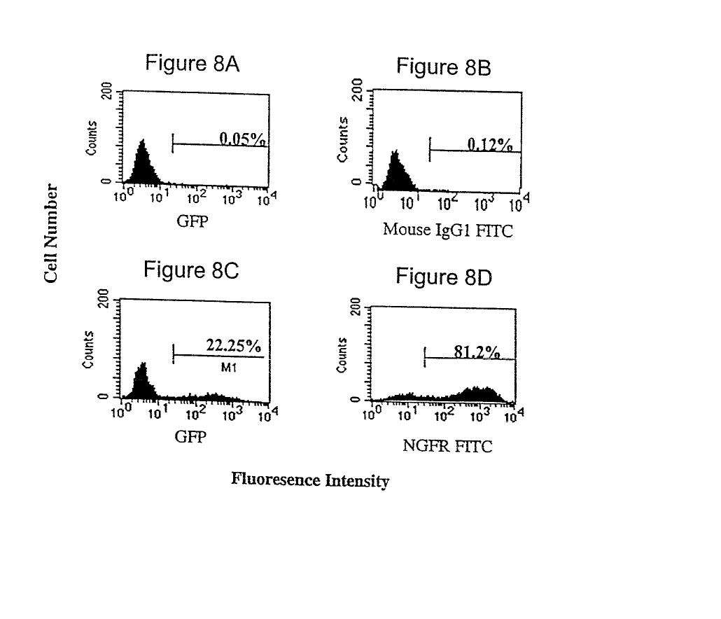

Next procedures were developed for efficiently transducing RBC precursors generated in the mononuclear cells cultures. In order to facilitate these studies, the Luv retroviral vector was developed (FIG. 7). Components of the Luv vector included: 1) utilization of an extended packaging signal which includes a portion of the gag coding sequence to enhance packaging of the viral genomic RNA and increase titer (Eglitis et al, Science 230(4732):1395-1398 (1985)), 2) utilization of the wild type MoMLV splice signals and the viral envelope start codon (ATG) for initiation of translation of vector encoded transgene to facilitate viral RNA processing and transgene expression (Sadelain et al, Proc. Natl. Acad. Sci. USA 92:6728-6732 (1995)) 3) disruption of the Pr65 gag open reading frame with a stop codon to minimize the possibility of translating gag peptides which could contribute to vector immunogenicity and toxicity, 4) elimination of any MoMLV envelope sequences which could contribute to vector immunogenicity and toxicity, and 5) inclusion of a multiple cloning site within the 3′ LTR U3 region for development of double copy vectors (Hantzopoulos et al, Proc. Natl. Acad. Sci. USA 86(10):3519-3523 (1989)). In order to identify cells transduced with Luv derived vectors, a truncated version of the Nerve Growth Factor Receptor (NGFR) gene as well as the optimized Green Fluorescence Protein (GFP) were cloned into the Luv vector to yield LuvNM and LuvGM respectively (Rudoll et al, Gene Therapy 3:695-705 (1996), McCowage et al, Experimental Hematology 26:288-298 (1998), Phillips et al, Nature Medicine 2:1154-1157 (1996)). These marker genes permit rapid determination of retroviral titer and evaluation of transduced cell populations using multiparameter FACS analysis. In addition, Luv was constructed in a modular manner so that important components of the vector could be easily exchanged in future studies with alternative sequences designed to enhance titer, stability and transgene expression. When transduced into NIH3T3 cells, both LuvNM and LuvGM demonstrated high level expression of the marker gene and were produced at titers 1-3×106 i.u./ml (FIG. 8).

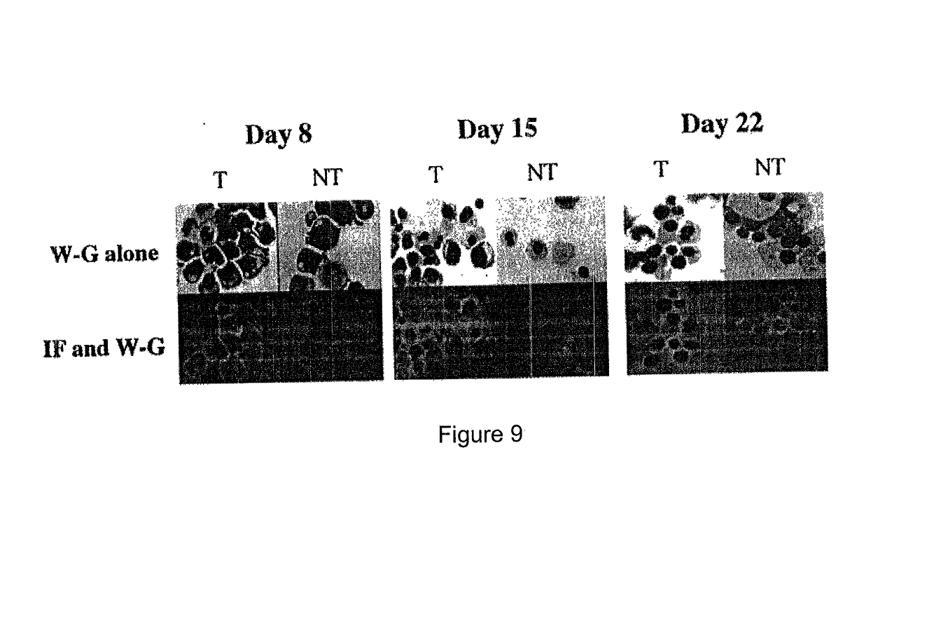

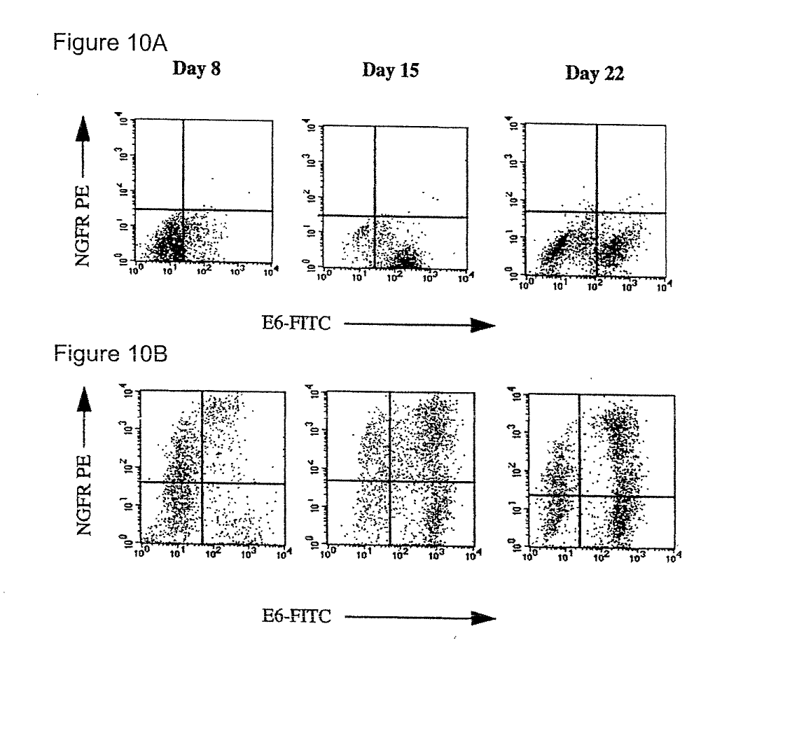

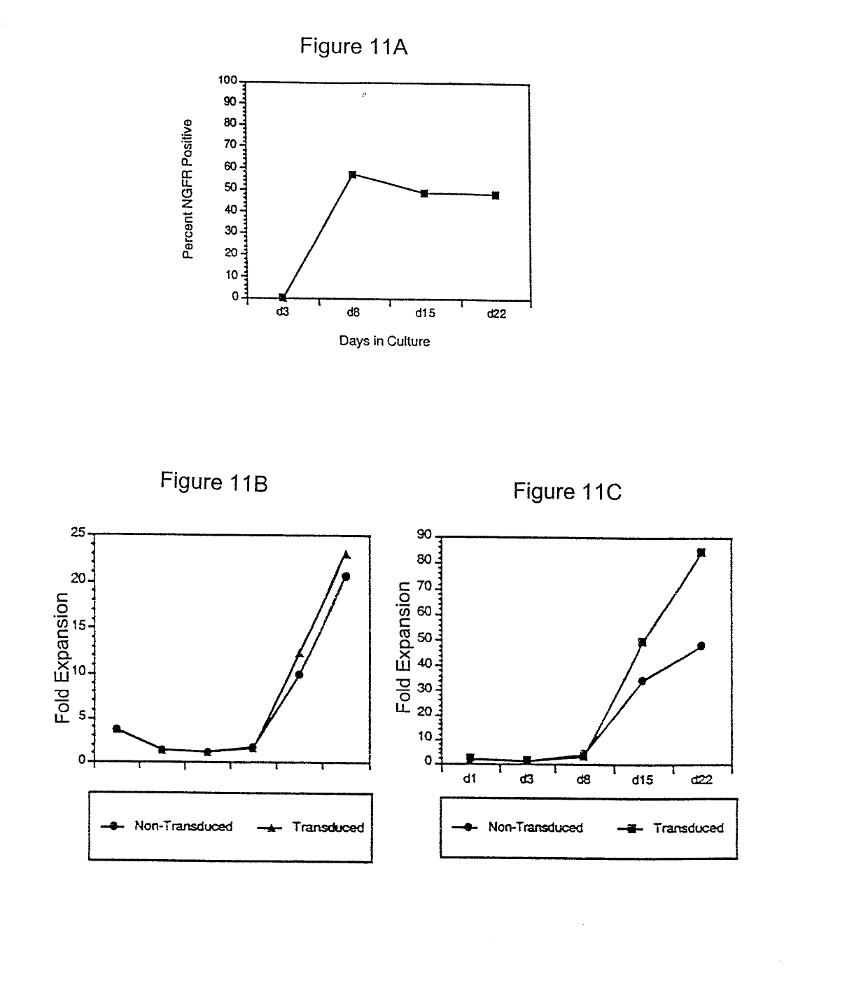

Using the Luv vectors, a variety of gene transfer procedures were evaluated in erythroid cells generated from UCB mononuclear cells. These studies demonstrated that the most efficient method was to transfer mononuclear cells onto culture plates previously coated with fibronectin fragments and then add retroviral supernatant daily for three days. Transduced erythroid cells could be easily recognized by immunofluorescent microscopy and FACS analysis (FIGS. 9 and 10). These techniques resulted in a mean tansduction efficiency of the erythroid cells of 51% (range 36.4-66%) (FIG. 11A). Expression of the marker gene remained consistent throughout the duration of the cultures indicating that stable gene transfer and transgene expression had occurred. In order to determine whether the vector or the transduction process altered the biology of the RBC precursors, UCB derived erythroid cells were transduced with the LuvNM vector and analyzed for total cell and erythroid cell growth. No difference was noted in the total fold expansion or erythroid lineage expansion of transduced cells relative to non-transduced cells (FIGS. 11B and 11C).

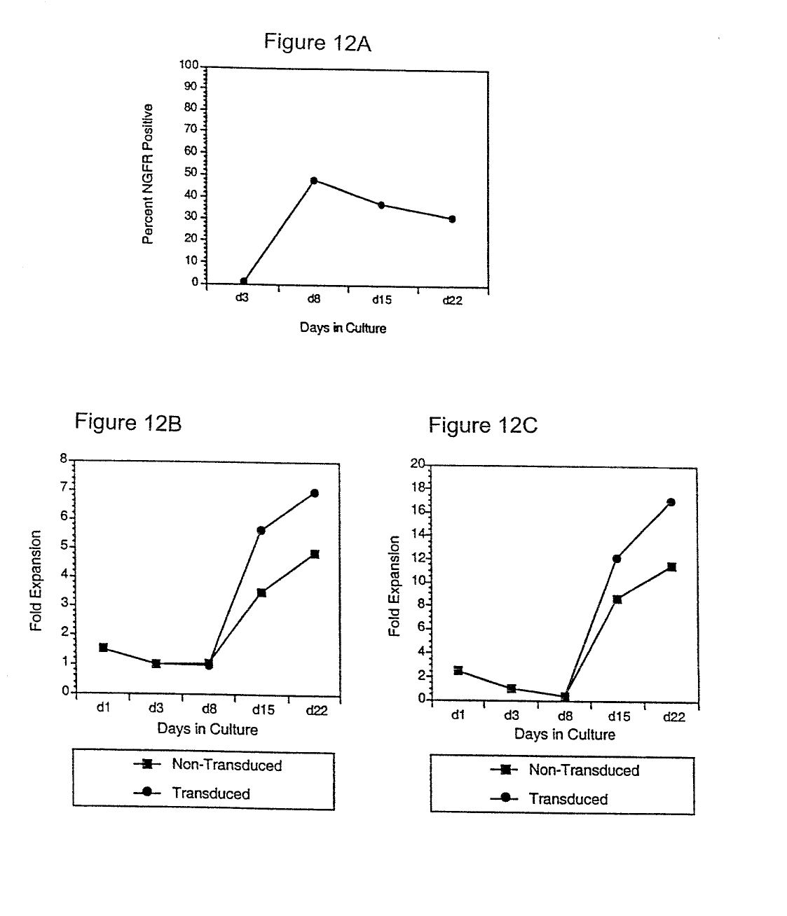

Next, these gene transfer procedures were evaluated in erythroid cells generated from PBMCs obtained from patients with Hb SC disease. While the gene transfer efficiency in the Hb SC erythroid cells was slightly lower than in UCB erythroid cells, a mean of 50% of the Hb SC erythroid cells were stably transduced (FIG. 12A). Again, the growth pattern of total cells and Hb SC erythroid lineage cells was unaffected by transduction with the LuvNM vector (FIGS. 12B and 12C). These observations indicate that erythroid cells generated from mononuclear cells obtained from both UCB and peripheral blood of patients with Hb SC disease can be efficiently and stably transduced with the Luv vector without obvious effects on erythroid biology.

All documents cited above are hereby incorporated in their entirety by reference.

One skilled in the art will appreciate from a reading of this disclosure that various changes in form and detail can be made without departing from the true scope of the invention.

What is claimed is:

1. A retroviral vector comprising, 5′ to 3′ and in operable linkage, a 5′ long terminal repeat (LTR), a splice donor, a packaging sequence, a gag open reading frame (ORF) mutated to reduce translation of gag peptides, a splice acceptor, a start codon in frame with a DNA sequence and a 3′ LTR.

2. The vector according to claim 1 wherein said vector further comprises a multiple cloning site (MCS) in said 3′ LTR.

3. The vector according to claim 1, wherein said packaging signal is an extended N2-derived packaging signal.

4. The vector according to claim 1 wherein said splice signals are wild type retroviral splice signals.

5. The vector according to claim 1 wherein said start codon is viral env ATG.

6. The vector according to claim 1 wherein said mutation in said gag ORF is an ATG to TAG mutation.

7. The vector according to claim 1 wherein said vector further comprises a polyadenylation signal 3′ to said 3′ LTR.

8. The vector according to claim 1 wherein said DNA sequence encodes an RNA antisense sequence or an RNA that is a DNA or RNA binding protein recognition sequence.

9. The vector according to claim 1 wherein said DNA sequence encodes an RNA antisense sequence.

10. The vector according to claim 9 wherein said RNA antisense sequence is complementary to a nucleotide sequence encoded in the genome of a pathogen.

11. The vector according to claim 10 wherein said pathogen is a bacteria or virus.

12. The vector according to claim 11 wherein said pathogen is human immunodeficiency virus.

13. The vector according to claim 1 wherein said DNA sequence encodes a polypeptide or protein.

14. The vector according to claim 13 wherein the polypeptide or protein is a mammalian polypeptide or protein.

15. The vector according to claim 1 wherein the DNA sequence encodes a selectable or identifiable phenotypic trait.

16. A method of producing an infectious viral particle comprising transfecting the retroviral vector of claim 1 into a retroviral packaging cell line under conditions such that said viral particle is produced, and recovering the viral particle.

17. A viral particle produced by the method of claim 16.

18. A packaging cell comprising the retroviral vector according to claim 1.

19. A method of introducing a transcription unit into a eucaryotic cell comprising infecting the cell with the viral particle according to claim 17.

20. The method according to claim 19 wherein said cell is a mammalian cell.

21. The method according to claim 20 wherein said cell is a human cell.

22. The method according to claim 21 wherein said cell is present in a human.

23. A isolated eucaryotic cell produced by the method of claim 19.

24. A pharmaceutical composition comprising the vector according to claim 1, a packaging cell comprising said vector or a viral particle produced by said packing cell, or a eucaryotic cell infected with said viral particle, and a carrier, diluent, adjuvant or excipient.
